(19) United States
(12) Patent Application Publication
(10) Pub. No.: US 2017/0308923 A1
(43) Pub. Date: Oct. 26, 2017

(54) EVALUATION APPARATUS AND EVALUATION METHOD

(71) Applicant: CENTAN INC., Tokyo (JP)

(72) Inventors: Takayasu Sekine, Itabashi-ku,Tokyo (JP); Tomomitsu Herai, Yokohama-shi, Kanagawa-ken (JP); Aya Hatano, Koto-ku, Tokyo (JP); Hiroshi Nittono, Higashihiroshima-shi, Hiroshima-ken (JP); Takanobu Tanaka, Chiyoda-ku, Tokyo (JP)

(73) Assignee: CENTAN INC., Tokyo (JP)

(21) Appl. No.: 15/533,685

(22) PCT Filed: Dec. 4, 2015
(86) PCT No.: PCT/JP2015/084149

§ 371 Date: Jun. 7, 2017

(30) Foreign Application Priority Data
Dec. 9, 2014 (JP) 2014-249272

(51) Int. Cl. G06Q 30/02; A61B 5/16; A61B 5/0484

(52) U.S. Cl. CPC G06Q 30/0242; G06Q 30/0272; A61B 5/04842; A61B 5/04845; A61B 5/16

(57) ABSTRACT

An evaluation apparatus comprises a brain wave acquiring unit that acquires a brain wave signal from each of a plurality of subjects; an intensity data generating unit that generates intensity data that represents an intensity of a signal component in a predetermined frequency band or a relation between intensities of signal components in a plurality of frequency bands; a correlation data generating unit that acquires pairs of two subjects for all combinations, calculates a cross-correlation coefficient for the intensity data at each point in time for the respective pairs, and generates, correlation data in the time-series form; and an output unit that generates evaluation data that numerically represents a degree of synchronization of brain wave fluctuation based on the correlation data.

### TECHNICAL FIELD

The present invention relates to an apparatus and a method for evaluating an object.

### BACKGROUND ART

A main method for advertisement aimed at consumers is TV commercials. TV commercials are broadcast in a limited time, for example, in 15 or 30 seconds, and thus, TV commercials are considered to be more preferable that more strongly impress consumers during the short-time viewing.

A method for evaluating a produced commercial is to make evaluators randomly extracted from general viewers view and subjectively evaluate the commercial.

However, the evaluators may not always accurately understand and express their states of mind. For example, the evaluators may not be always accurately aware of a high marketing effect of a video even though the video actually has such an effect. Thus, the mere subjective evaluation is considered to be insufficient to produce fair results.

A technique for objectively evaluating a video, an image, or the like is, for example, an evaluation method described in PTL 1. In the evaluation method, an evaluation object (for example, a design of a new product) is shown to an evaluator whose brain waves are being measured. Pattern matching is performed on the measured brain waves to estimate what feeling the evaluator has in viewing the object. PTL 1 states that the method described therein allows production of more intuitive evaluation results that are independent of subjects' subjective views.

### CITATION LIST

### Patent Literature

### [PTL 1] Japanese Patent Application Laid-open No. 2004-342119

### SUMMARY OF INVENTION

### Technical Problem

In the evaluation method described in PTL 1, a pattern of a brain wave potential acquired from the subject is matched against a plurality of pre-stored patterns to estimate the subject's feeling. However, the human brain wave contains components with various frequencies, and does not show characteristic reactions in association with particular feelings. That is, it is difficult to estimate the subject's feeling simply by acquiring the brain potential.

On the other hand, a technique is well known in which frequency analysis is executed on the acquired brain waves to obtain the intensities of signal components such as an alpha wave and a theta wave which have particular meanings. However, these frequency components also do not allow observation of absolute patterns based on particular feelings.

The present invention has been achieved in view of the above-described problems. An object of the present invention is to provide an evaluation apparatus that evaluates an object using a brain wave acquired from a subject.

### Solution to Problem

The present invention in its one aspect provides an evaluation apparatus comprising a brain wave acquiring unit configured to acquire a brain wave signal acquired from each of a plurality of subjects; an intensity data generating unit configured to generate, based on the brain wave signal, intensity data that represents, in a time-series form, an intensity of a signal component in a predetermined frequency band or a relation between intensities of signal components in a plurality of frequency bands; a correlation data generating unit configured to acquire pairs of two subjects of the plurality of subjects for all combinations, calculate a cross-correlation coefficient for the intensity data at each point in time for the respective pairs, and generate, for the respective pairs, correlation data that represents the cross-correlation coefficients in the time-series form; and an output unit configured to generate evaluation data that numerically represents a degree of synchronization of brain wave fluctuation among all of the plurality of subjects based on the correlation data generated for all the pairs.

The brain wave acquiring unit is means for acquiring the brain wave signals acquired from the plurality of subjects. The brain wave signals may be acquired from electroencephalographs installed on the subjects or may be pre-measured, saved, and acquired. Each of the brain wave signals is data indicative of a detected brain potential.

Furthermore, the intensity data generating unit is means for generating data (intensity data) indicative of the intensity of the signal component in the predetermined frequency band based on the acquired brain wave signal. The predetermined frequency band may be a frequency band frequently used for analysis in the field of neuroscience, such as a band corresponding to an alpha wave, a beta wave, a gamma wave, or a theta wave, respectively, or any frequency band.

Furthermore, the intensity data may be the relation between the intensities of the signal components in the plurality of frequency bands, such as the ratio of the intensity in the alpha band to the intensity in the beta band.

The intensity data varies, for example, due to excitement and tension of the brain, the degree of concentration in the brain, and the like. However, directly evaluating the subject's state based on single intensity data is difficult. Thus, in the evaluation apparatus according to the present invention, the correlation data generating unit generates, for all pairs of subjects, the data (correlation data) indicative of the degree of correlation of the intensity data among the subjects of each subject pair. The generation of the correlation data allows acquisition of data indicating the degree to which brain wave fluctuation is synchronous among a plurality of subjects.

For example, when the correlation data indicates a stronger correlation, this means that brain wave fluctuation in is synchronous among the subjects, that is, the waveforms of the intensity data are similar.

The thus generated data is data having an evaluation scale corresponding to the target frequency band. For example, when the target frequency band is a frequency band in which the intensity varies according to the degree of the subject's concentration, if a phenomenon in which a strong correlation is exhibited between the intensity data is observed in many pairs, this indicates that many subjects concentrate their awareness.

The output unit is means for generating and outputting the data (evaluation data) numerically representing, for each point in time, the degree of synchronization of brain wave fluctuation in among the subjects based on the generated correlation data. The output data may represent the degree of synchronization for all of the plurality of subjects or for particular subject pairs. Furthermore, the output may be numerical values or images or the like.

As described above, the evaluation apparatus according to the present invention executes frequency analysis on the brain wave signals acquired from the plurality of subjects, and outputs evaluation results based on the degree of synchronization of fluctuation in signal intensity among the subjects. This enables acquisition of information that fails to be observed based on a brain wave obtained from a single subject.

Also, the intensity data generating unit may generate the intensity data for each of a plurality of frequency bands, the correlation data generating unit may generate the correlation data for each of the plurality of frequency bands, and the output unit may generate and output the evaluation data for each of the plurality of frequency bands.

In general, human brain waves are considered to contain components in various frequency bands having different meanings. Therefore, evaluation results having different meanings can be obtained by analyzing each of the signal components in the plurality of different frequency bands.

Also, the predetermined frequency band may be a frequency band in which an intensity of a signal component in the frequency band varies due to excitement of senses including a visual sense and an auditory sense or concentration of attention.

This allows evaluation of the degree to which the subject is excited or attracted through a sense such as a visual sense or an auditory sense.

Also, brain wave signals acquired by the brain wave acquiring unit may be brain wave signals acquired from the plurality of subjects made to view the same video or listen to the same sound.

The evaluation apparatus according to the present invention can be suitably used as an apparatus that objectively evaluates a material such as a video or a sound.

Also, the output unit may generate, at each point in time, an image that represents a degree of correlation of the intensity data among a plurality of the subjects, in hues or brightness for respective pairs of subjects, and output as a moving image together with the video that the subjects are made to view or the sound to which the subjects are made to listen.

As described above, the degree of the correlation of the intensity data is represented by hues or brightness for respective pairs of subjects. This allows a timing when brain wave fluctuation is likely to be synchronous to be determined at a glance.

Also, the output unit may generate a sound signal that represents a degree of correlation of the intensity data among a plurality of the subjects, based on variation in pitch or volume, and output the sound signal along with the video that the subjects are made to view or the sound to which the subjects are made to listen.

As described above, the sound signal may be used for aural notification of the timing when brain wave fluctuation is likely to be synchronous.

The present invention may be identified as an evaluation apparatus including at least some of the above-described means.

Furthermore, the present invention may be identified as an evaluation method executed by the evaluation apparatus. The above-described processing and means may be freely combined together unless the combination results in technical inconsistency.

### Advantageous Effects of Invention

The present invention may provide an evaluation apparatus that evaluates an object using a brain wave acquired from a subject.

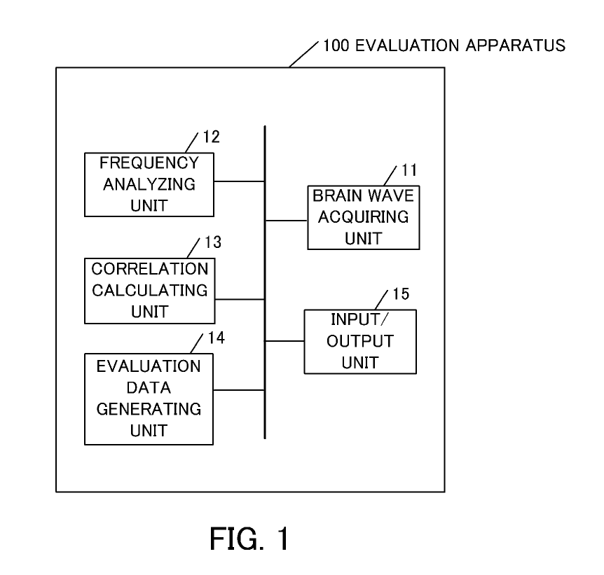

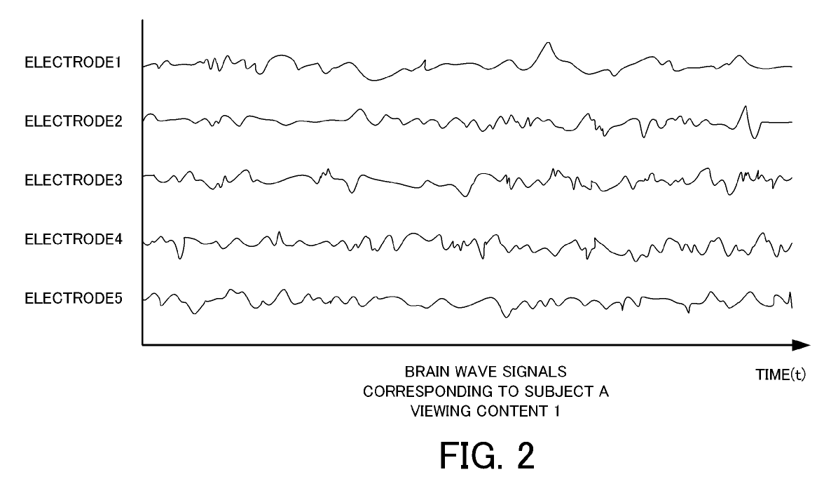

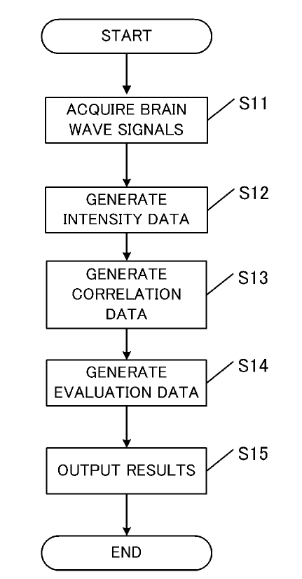

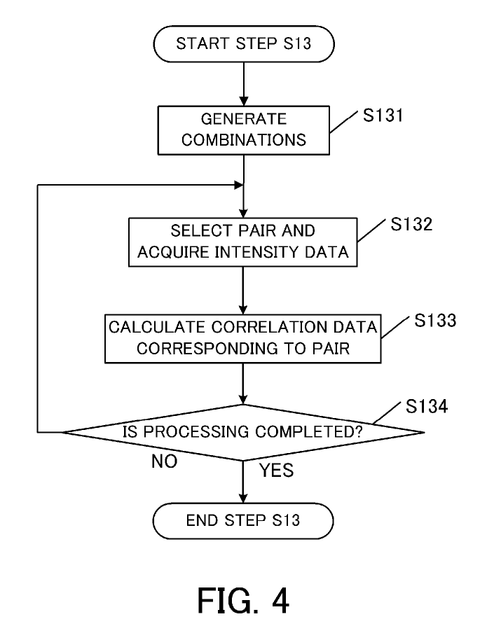

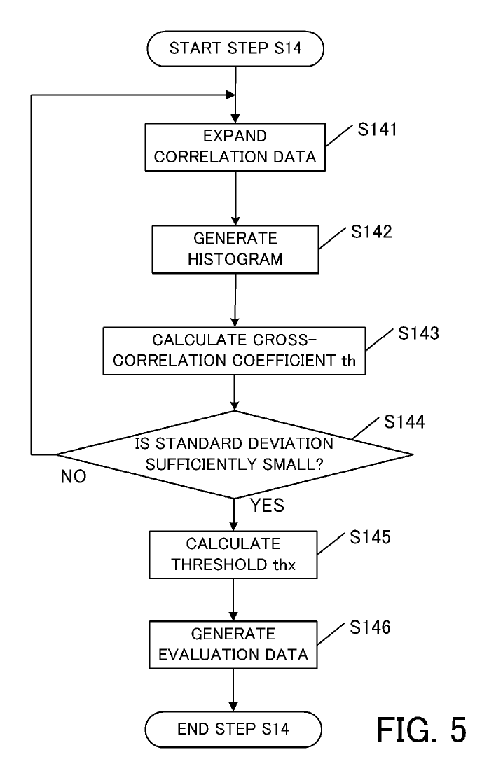

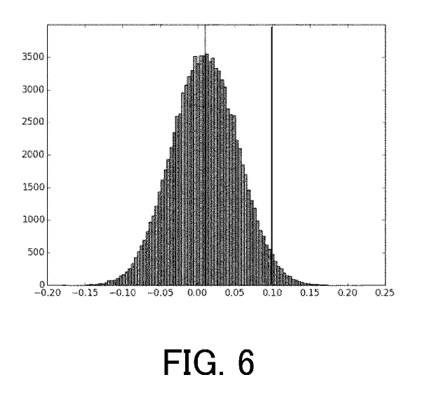

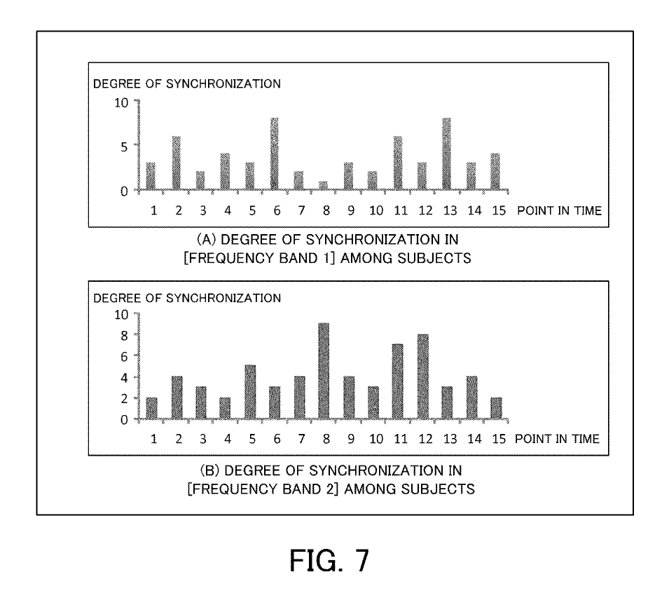

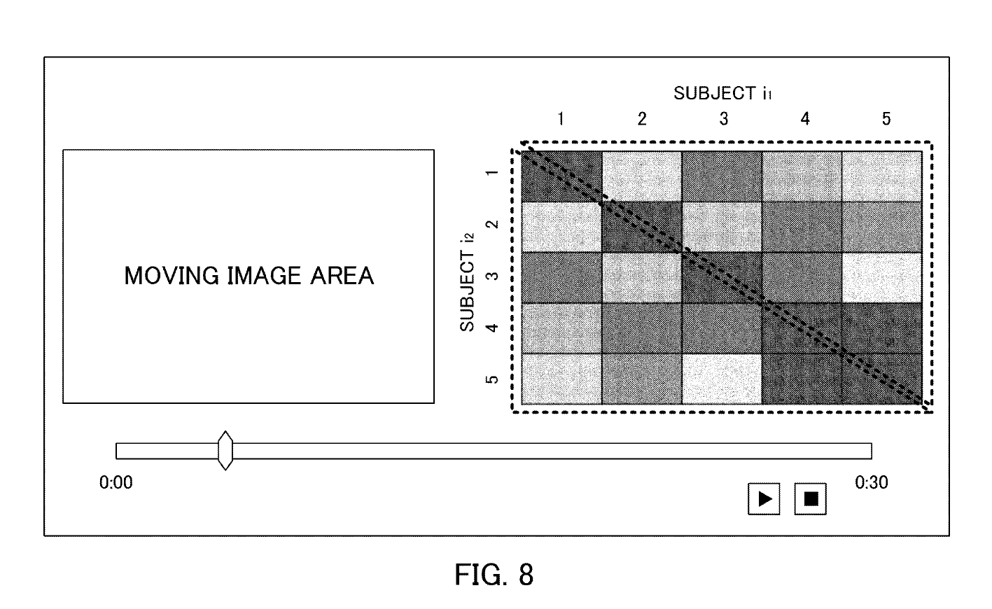

### BRIEF DESCRIPTION OF DRAWINGS

[FIG. 1] FIG. 1 is a diagram of a system configuration of an evaluation apparatus according to a first embodiment.

[FIG. 2] FIG. 2 is a diagram of an example of brain wave signals.

[FIG. 3] FIG. 3 is a flowchart of processing executed by an evaluation apparatus 100.

[FIG. 4] FIG. 4 is a second flowchart of processing executed by the evaluation apparatus 100.

[FIG. 5] FIG. 5 is a third flowchart of processing executed by the evaluation apparatus 100.

[FIG. 6] FIG. 6 is an example histogram generated in step S142.

[FIG. 7] FIG. 7 is an example of evaluation results output in a first embodiment.

[FIG. 8] FIG. 8 is an example of evaluation results output in a fourth embodiment.

### DESCRIPTION OF EMBODIMENTS

### First Embodiment

Preferred embodiments of the present invention will be described below with reference to the drawings. An evaluation apparatus 100 according to a first embodiment is an apparatus that generates evaluation of an evaluation object based on acquired brain wave signals. Furthermore, the brain wave signals acquired by the evaluation apparatus 100 are obtained by measuring the brain waves of a plurality of subjects while making the subjects view an evaluation object.

### <System Configuration>

FIG. 1 is a diagram of a system configuration of the evaluation apparatus 100 according to the first embodiment. The evaluation apparatus 100 includes a brain wave acquiring unit 11, a frequency analyzing unit 12, a correlation calculating unit 13, an evaluation data generating unit 14, and an input/output unit 15.

The brain wave acquiring unit 11 is means for acquiring a brain wave signal to be analyzed. The brain wave signals acquired by the brain wave acquiring unit 11 are obtained from a plurality of subjects using measurement means such as electroencephalographs.

Now, the brain wave signals to be acquired will be described in brief. In general, to acquire human brain waves, a plurality of electrodes is arranged on a subject's scalp and potentials (brain potentials) obtained through the plurality of electrodes are collected. The brain wave signal is hereinafter assumed to be time-series data indicative of the brain potential for each electrode.

The positions where the electrodes are arranged may be, for example, those specified in the International Law 10-20, which is generally used, or may be in any other form. For example, if a certain characteristic is known to appear at a particular position in a concentrative manner, the electrodes may be concentrated at the particular position (for example, the frontal region of the head).

The brain wave signal acquired by the brain wave acquiring unit 11 is obtained by pre-measuring, using an electroencephalographs, the brain wave of a subject to which a commercial video to be evaluated (hereinafter referred to as a content) is shown. The target brain wave signal may be acquired by loading the corresponding data stored in a storage medium or via a network or the like. Of course, the brain wave signal may be acquired in real time using an electroencephalograph installed on the subject.

The brain wave signal is sampled every predetermined time. For example, if a content lasts 30 seconds and a sampling frequency is 100 Hz, 3,000 time steps are provided. The term “point in time” is used as a time that has elapsed since a reproduction start time for the content, which is defined to be 0. FIG. 2 is a diagram illustrating brain wave signals obtained from a certain subject viewing a certain content.

The frequency analyzing unit 12 is means for executing frequency analysis on the brain wave signal acquired by the brain wave acquiring unit 11 to generate an intensity signal for a predetermined band (intensity data in the present invention). The predetermined band is selected from frequency bands frequently used for analysis in the field of neuroscience, for example, a theta wave (4 Hz or higher and lower than 8 Hz) and an alpha wave (8 Hz or higher and lower than 13 Hz). Alternatively, any band may be used according to the properties of the brain wave signal.

The intensity data obtained is data in a time-series form that is indicative of the intensity of a signal component.

The correlation calculating unit 13 is means for generating data (correlation data in the present invention) indicative of a cross-correlation between a plurality of intensity data generated by the frequency analyzing unit 12. The correlation data is obtained by representing a cross-correlation coefficient between intensity data calculated for each point in time, in the time-series form. An example of the correlation data and a detailed method for calculating the correlation data will be described below.

The evaluation data generating unit 14 is means for generating evaluation data to be presented to a user of the apparatus, that is, evaluation results for the content, based on the correlation data generated by the correlation calculating unit 13. An example of the evaluation data and a detailed method for calculating the evaluation data will be described below.

Control of each of the above-described means is achieved by a processing device such as a CPU executing a control program. Furthermore, the control may be achieved by an FPGA (Field-Programmable Gate Array), an ASIC (Application Specific Integrated Circuit), and the like, or a combination thereof. Alternatively, the control may be achieved by dedicated hardware.

### <Process Flowchart>

Now, the contents of a specific process executed by the means will be described with reference to FIG. 3 that is a process flowchart. The process illustrated in FIG. 3 is started at a timing when the user (operator) of the apparatus gives an instruction to start analysis.

First, in step S11, the brain wave acquiring unit 11 acquires pre-measured brain wave signals. The brain wave signals acquired herein are data represented in the time-series form and indicating a brain potential for each electrode and for each subject. In the present embodiment, the brain wave signal is denoted as S_{i,e,h}(t). In this case, i denotes a subject number, e denotes an electrode number, h denotes a content number, and t denotes a point in time. The brain wave signals acquired are transmitted to the frequency analyzing unit 12.

In step S12, the frequency analyzing unit 12 executes Fourier transform on the brain wave signals to generate data (intensity data) indicative of the intensity of a signal component in a predetermined frequency band. In the present embodiment, the intensity data is denoted as PF_{i,e,h}(t). The meanings of i, e, h, t are the same as those for the brain wave signals.

In the present embodiment, Fourier transform is used as a frequency analysis technique. However, any other frequency analysis technique may be used, for example, wavelet transform or complex demodulation.

For an increased calculation speed, a process for reducing the amount of data may be executed before the frequency analysis is performed. For example, downsampling may be performed after the brain wave signals are acquired at a sampling rate higher than a target sampling rate. For example, downsampling may be performed at 200 Hz after sampling is executed at 1000 Hz.

Furthermore, a process may be executed in which signal components (artifacts) attributed to human body activities irrelevant to thoughts are detected and canceled. The brain wave may be varied by human body activities such as breathing and blinking. Thus, addition of the process for canceling such components allows accuracy to be improved.

A typical technique for removing artifact components from the brain wave is an independent component analysis (hereinafter referred to as the ICA). The ICA is an analysis technique for separating multivariate data into a plurality of additive components. This allows each acquired brain wave signal to be separated into a plurality of components so as to enable removal of components irrelevant to brain activities related to thoughts, feelings, senses, and the like, for example, components attributed to a blink and body motion. Furthermore, after the artifact components are removed, a reverse process is executed to reconstruct the brain wave signal. This allows the brain wave signal with reduced artifact components to be obtained.

A known technique may be used to detect artifact components. For example, the kurtosis of the brain wave signal may be determined to detect an instantaneous motion such as a blink. Alternatively, the artifact components may be detected using learning data or the like. Furthermore, if artifact components are generated at particular electrodes in a biased manner, the positions of the electrodes may be taken into account.

The intensity data generated by the frequency analyzing unit 12 is transmitted to the correlation calculating unit 13.

In step S13, the correlation calculating unit 13 generates correlation data indicative of the correlation between a plurality of intensity data. FIG. 4 is a diagram illustrating processing executed in step S13 in further detail.

First, in step S131, combinations of the plurality of subjects are generated. For example, for n subjects, ₙC₂ combinations are possible.

Then, in step S132, one unprocessed pair is selected from the generated combinations, and the corresponding intensity data is acquired. For example, if a pair with subject numbers 1 and 2 is selected, PF_{1,e,h}(t) and PF_{2,e,h}(t) are acquired.

Then, in step S133, the correlation data corresponding to the selected pair is calculated. Specifically, a window for calculation (unit interval) is set in the intensity data, and with the window slid, the cross-correlation coefficient is calculated at each point in time. Finally, a row of cross-correlation coefficients represented in the time-series form is generated as correlation data. Equation (1) is an equation that allows the cross-correlation coefficient between the intensity data to be calculated.

In the equation, i1 denotes the subject number of the first subject, and i2 denotes the subject number of the second subject. Furthermore, barred items are the average values of intensities. Additionally, w+1 denotes a window width.

$$\begin{matrix}[ Math . 1 ] & \\ {CR}_{i 1 , i 2 , e , h} ( t ) = \frac{{\sum }_{t - \frac{w}{2}}^{t 1 \frac{w}{2}} ( {PF}_{i 1 , e , h} ( t ) - \overline{{PF}_{i 1 , e , h} ( t )} ) ( {PF}_{i 2 , e , h} ( t ) - \overline{{PF}_{i 2 , e , h} ( t )} )}{\sqrt{{\sum }_{{t}_{2}^{w}}^{t {1}_{2}^{w}} {( {PF}_{i 1 , e , h} ( t ) - \overline{{PF}_{i 1 , e , h} ( t )} )}^{2}} \sqrt{{\sum }_{{t}_{2}^{w}}^{t {1}_{2}^{w}} {( {PF}_{i 2 , e , h} ( t ) - \overline{{PF}_{i 2 , e , h} ( t )} )}^{2}}} & Equation ( 1 )\end{matrix}$$

Correlation data CR calculated in accordance with Equation (1) is indicative of the correlation of the intensity data between the subject numbers i1 and i2 represented in the time-series form for each electrode and for each content.

Then, in step S134, whether the above-described process has been executed on all combinations of the subjects is checked. If the process has not been completed, the process is shifted to step S132 where the next pair is selected and the process is continued. If the process has been executed on all the combinations, the processing in step S13 is ended.

The correlation data generated by the correlation calculating unit 13 is transmitted to the evaluation data generating unit 14.

With reference back to FIG. 3, description will be continued.

In step S14, the evaluation data generating unit 14 generates an evaluation of the content based on the calculated correlation data. FIG. 5 is a diagram illustrating processing executed in step S14 in further detail.

The evaluation apparatus according to the present embodiment evaluates a content by determining whether the brain waves of a plurality of subjects are synchronous based on the correlation data generated by the correlation calculating unit 13. In this case, an increase in evaluation accuracy needs a determination of whether the correlation acquired by the correlation calculating unit 13 has been accidentally obtained or has resulted from synchronization of the brain waves due to viewing of the content. Thus, in step S14, first, a process for calculating a threshold for the above-described determination (steps S141 to S145) is executed based on the correlation data generated by the correlation calculating unit 13. Data allowing the content to be evaluated is generated using the threshold (step S146). The processing executed in steps S141 to S146 will be described below.

First, in step S141, the correlation data generated by the correlation calculating unit 13, that is, CR_{i1,i2,e,h}(t), is acquired, and all the data (ₙC₂×e×h×t data. In this case, n is the number of subjects) are expanded.

Then, in step S142, as many records as the pairs of subjects (ₙC₂ records) are randomly extracted from all the data in all the intervals resulting from the expansion, and an average value is calculated for the records. For example, for 15 subjects, 105 pairs of the subjects are possible. Thus, 105 cross-correlation coefficients are extracted, and the average value of the cross-correlation coefficients is calculated. This operation is repeated a predetermined number of times (for example, 100,000 times), and the frequency of the average value obtained is represented as a histogram. FIG. 6 is an example of a histogram generated in step S142.

Then, in step S143, a cross-correlation coefficient th₁ corresponding to the top x% of the generated histogram is calculated. That is, the value is determined at which the probability that the cross-correlation coefficient is equal to or smaller than th1 is (100−x)%. In this case, x may be, for example, 1% but may be any other value.

The processing in steps S141 to S143 described above is executed a plurality of times in a loop to collect a plurality of cross-correlation coefficients (th1, th2, th3 . . . ), and the loop is ended when the standard deviation among the plurality of cross-correlation coefficients is sufficiently small (step S144). Finally, the average is taken for the acquired plurality of cross-correlation coefficients as a threshold thₓ (step S145).

Step S146 is a step in which time-series data indicative of the degree of the correlation of brain wave fluctuation between the subjects is generated using the correlation data generated by the correlation calculating unit 13, that is, CR_{i1,i2,e,h}(t), and the threshold thₓ generated in step S145. The data thus generated is indicative of an evaluation of the content.

The present step is a step in which the particular content is evaluated, and thus, h is fixed (this is omitted in the equation).

In step S146, processing described below is executed.

First, for the target content, the average value of the cross-correlation coefficients for all the subject pairs is calculated for each electrode and for each analysis interval in accordance with the following Equation 2.

$$\begin{matrix}[ Math . 2 ] & \\ \overline{{CR}_{e} ( t )} = \frac{1}{{ }_{n} {C}_{2}} {\sum }_{i , j \\ ( i + j )} {CR}_{i , j , e} ( t ) & Equation ( 2 )\end{matrix}$$

A distance Δₑ(t) between the calculated distance and the thₓ calculated in step S141 is calculated in accordance with the following Equation (3).

[Math. 3]

Δₑ(t)=C̅R̅ₑ̅(̅t̅)̅−thₓ Equation (3)

Then, the calculated Δₑ(t) is fitted to a logistic function represented by Equation (4) illustrated below to calculate Δₑ(t). In the equation, v is a positive constant (for example, v=1, v→∞).

$$\begin{matrix}[ Math . 4 ] & \\ {A}_{e} ( t ) = {1}_{1 + {e}^{- v Δ ( t )}} & Equation ( 4 )\end{matrix}$$

The resultant Aₑ(t) is hereinafter referred to as degree-of-synchronization data. The degree-of-synchronization data is time-series data resulting from weighting of the correlation data based on thx. The value of Aₑ(t) is indicative of the degree of synchronization, and a larger value means that fluctuation in intensity data is more synchronous among a plurality of subjects.

The degree-of-synchronization data calculated in step S14 is represented as a graph, which is provided via the input/output unit 15 to the user of the apparatus as evaluation data in the present invention.

The output degree-of-synchronization data may be data for each electrode or may be integrated data for all the electrodes. Calculation may be executed to obtain data for a plurality of frequency bands, an intensity ratio between the frequency bands, and the like, and a plurality of graphs obtained may be simultaneously output. FIG. 7 is an example screen resulting from simultaneous output of a graph of the degree-of-synchronization data corresponding to a frequency band 1 and a graph of the degree-of-synchronization data corresponding to a frequency band 2.

The above-described embodiment provides numerical values indicative of the degree to which fluctuation in the intensity of the predetermined frequency component of the brain wave is synchronous among the plurality of subjects. A high degree of synchronization means that a plurality of subjects undergoes similar reactions, thus allowing evaluation of the degree to which the content impacts the subjects' awareness, unawareness, and senses. For example, the embodiment allows numerical evaluation of, for example, the degree to which the target content impacts the subjects or makes the subjects' awareness concentrate.

Furthermore, varying the target frequency band enables evaluation using various criteria.

Additionally, when a graph in the time-series form is output as evaluation data, the timing when the subjects undergo reactions can be determined.

### Second Embodiment

In the first embodiment, the degree-of-synchronization data in the time-series form is output as the evaluation data. In contrast, a second embodiment is an embodiment in which a single evaluation value is calculated for a target content and output as evaluation data. A system configuration of an evaluation apparatus according to the second embodiment is similar to the system configuration in the first embodiment. Thus, detailed description of the system configuration of the second embodiment is omitted, and only differences in processing between the first and second embodiments will be described.

In the second embodiment, after step S146 is completed, a step is executed to calculate a single evaluation value corresponding to the content based on the degree-of-synchronization data resulting from step S146.

First, with an electrode number e fixed and a time step t varied, the degree-of-synchronization data Aₑ(t) is multiplied by a weight defined for each time step, and results obtained are summed. This is executed for each electrode.

Then, with the electrode number e varied, the above-described result is multiplied by a weight defined for each electrode, and results obtained are summed to determine a final evaluation value Q.

The resultant value Q is an evaluation value for the content (hereinafter referred to as a content evaluation value). The calculated content evaluation value is provided to the user of the apparatus through the input/output unit 15.

The weight for each electrode is utilized in order to add different degrees of importance to the respective electrodes. For example, if a region is known in which a brain wave corresponding to a particular frequency band characteristically appears, the region may be provided with a heavier weight. Of course, the weight for each electrode may be 1.

Furthermore, the weight for each time step is utilized in order to add different degrees of importance to the respective time steps. For example, particularly for a scene that is to appeal to viewers or a scene that is to have an impact on the viewers, the weight for the corresponding time step may be increased. Of course, the weight for each time step may be 1.

In the above-described example, after the multiplication by the weight, the sum is determined twice. However, an infinite product may be used. Alternatively, the sum and the infinite product may each be used once. For example, for a short content, the evaluation value may be more sensitively calculated by using the infinite product rather than the sum. Alternatively, four evaluation value patterns (the sum and the sum, the sum and the infinite product, the infinite product and the sum, and the infinite product and the infinite product) may be calculated, and the resultant four evaluation values may be weighted and summed to obtain the final evaluation value.

This allows contents with varying properties to be optimally evaluated.

As described above, in the second embodiment, different weights are applied for the respective electrodes and for the respective time steps, and a single evaluation value is calculated. Consequently, a quantitative evaluation value may be obtained for the content.

### Third Embodiment

In the first and second embodiments, the evaluation results for the content are graphically or numerically output. In contrast, a third embodiment is an embodiment in which the degree of synchronization of brain wave fluctuation among the subjects is chromatically indicated.

In the third embodiment, after the processing in step S13 is completed, the processing in step S14 is omitted, and the process shifts to step S15. Furthermore, in step S15, for respective pairs of subjects, a display screen is generated based on the correlation data generated in step S13.

FIG. 8 is an example of a screen presented to the user in the third embodiment. As illustrated in FIG. 8, in the third embodiment, an N×N matrix is generated that represents pairs of subjects i1 and i2, and N² cells are used to express the corresponding cross-correlation coefficients. (N is the number of subjects.)

Specifically, first, for the correlation data (CR_{i1,i2,e,h}(t)) generated in step S13, a process is executed in which values corresponding to a plurality of electrodes are averaged. In this case, h is fixed and omitted from the equation. As a result, CR_{i1,i2}(t) is obtained.

Then, a color determined from the cross-correlation coefficient is assigned to the cell of the corresponding pair. Specifically, the numerical value is converted into a hue such that CR_{i1,i2}(t) with a negative value is converted into a cool color, whereas CR_{i1,i2}(t) with a positive color is converted into a warm color. For example, as the cross-correlation coefficient increases, dark blue, blue, pale blue, green, yellow, orange, red, and dark red may be assigned to the cross-correlation coefficient in this order.

Executing this processing on all the time steps allows the hues of the N² cells to be determined for all the time steps. Then, the corresponding image is generated for each time step, and a moving image is generated from the plurality of resultant images.

In the present embodiment, the cross-correlation coefficients are expressed in hues. However, the cross-correlation coefficients may be expressed in brightness or the like.

In the third embodiment, the moving image thus generated is reproduced simultaneously with reproduction of the content viewed by the subjects. This enables visual presentation of the degree to which brain wave fluctuation is synchronous among the subjects.

In the matrix depicted in FIG. 8, both an upper right area and a lower left area represent the same pairs of subjects, and thus, the hues appearing in the upper right area and in the lower left area are symmetric. However, different pieces of information may be output to the respective areas. For example, if analysis is conducted for two different frequency bands, results corresponding to a first frequency band may be output to the upper right area, whereas results corresponding to a second frequency band may be output to the lower left area.

### (Variations)

The above-described embodiments are illustrative and the present invention may be varied in implementation without departing from the spirits of the invention. For example, the illustrated embodiments may be combined together.

Furthermore, the technique for calculating the correlation data is not limited to the technique represented by Equation (1) and any technique may be used.

Additionally, in each of the embodiments, the correlation data is generated for respective pairs of subjects. However, information on three or more subjects may be integrated together to obtain correlation data indicative of the correlation among the three or more subjects.

In addition, in the first and second embodiments, the evaluation data is generated based on the degree-of-synchronization data. However, the execution of step S14 may be omitted and the correlation data may be output in any manner as an indicator used to evaluate the content. That is, the correlation data may be output as the evaluation data. Furthermore, in this case, the correlation data generated for respective pairs of subjects and for each electrode may be integrated together in any manner.

Additionally, in each embodiment, the evaluation data is visually output using a graph or hues. However, the evaluation data may be output using sound signals. For example, the pitch or volume of the sound signal may be proportional to the magnitude of the value of the degree-of-synchronization data or the correlation data. The sound signal is synchronized with the content so as to be reproduced simultaneously with reproduction of the content, to enable aural presentation of the degree to which brain wave fluctuation is synchronous among the subjects.

### REFERENCE SIGNS

100 Evaluation apparatus
11 Brain wave acquiring unit
12 Frequency analyzing unit
13 Correlation calculating unit
14 Evaluation data generating unit
15 Input/output unit

1. An evaluation apparatus comprising:
a brain wave acquiring unit configured to acquire a brain wave signal acquired from each of a plurality of subjects;
an intensity data generating unit configured to generate, based on the brain wave signal, intensity data that represents, in a time-series form, an intensity of a signal component in a predetermined frequency band or a relation between intensities of signal components in a plurality of frequency bands;
a correlation data generating unit configured to acquire pairs of two subjects of the plurality of subjects for all combinations, calculate a cross-correlation coefficient for the intensity data at each point in time for the respective pairs, and generate, for the respective pairs, correlation data that represents the cross-correlation coefficients in the time-series form; and
an output unit configured to generate evaluation data that numerically represents a degree of synchronization of brain wave fluctuation among all of the plurality of subjects based on the correlation data generated for all the pairs.

2. The evaluation apparatus according to claim 1, wherein
the intensity data generating unit generates the intensity data for each of a plurality of frequency bands,
the correlation data generating unit generates the correlation data for each of the plurality of frequency bands, and
the output unit generates and outputs the evaluation data for each of the plurality of frequency bands.

3. The evaluation apparatus according to claim 1, wherein the predetermined frequency band is a frequency band in which an intensity of a signal component in the frequency band varies due to excitement of senses including a visual sense and an auditory sense or concentration of attention.

4. The evaluation apparatus according to claim 1, wherein brain wave signals acquired by the brain wave acquiring unit are brain wave signals acquired from the plurality of subjects made to view the same video or listen to the same sound.

5. The evaluation apparatus according to claim 4, wherein the output unit generates, at each point in time, an image that represents a degree of correlation of the intensity data among a plurality of the subjects, in hues or brightness for respective pairs of subjects, and outputs as a moving image together with the video that the subjects are made to view or the sound to which the subjects are made to listen.

6. The evaluation apparatus according to claim 4, wherein the output unit generates a sound signal that represents a degree of correlation of the intensity data among a plurality of the subjects, based on variation in pitch or volume, and outputs the sound signal along with the video that the subjects are made to view or the sound to which the subjects are made to listen.

7. An evaluation method comprising:
a brain wave acquiring step of acquiring a brain wave signal acquired from each of a plurality of subjects;
an intensity data generating step of generating intensity data that represents, in a time-series form, an intensity of a signal component in a predetermined frequency band, based on the brain wave signal;
a correlation data generating step of acquiring pairs of two subjects of the plurality of subjects for all combinations, calculating a cross-correlation coefficient for the intensity data at each point in time for the respective pairs, and generating, for the respective pairs, correlation data that represents the cross-correlation coefficients in the time-series form;
an output step of generating evaluation data that numerically represents a degree of synchronization of brain wave fluctuation among all of the plurality of subjects, based on the correlation data generated for all the pairs.

8. A non-transitory computer readable storing medium recording a computer program for causing a computer to perform the evaluation method according to claim 7.
